(19) United States
(12) Patent Application Publication
(10) Pub. No.: US 2019/0172586 A1
(43) Pub. Date: Jun. 6, 2019

(54) SYSTEM AND METHOD FOR DETERMINING MEDICAL RISK FACTORS FOR PATIENTS

(71) Applicant: Predict Disease Private Limited, Ahmedabad (IN)

(72) Inventor: Shail S. CHOKSI, North Brunswick, NJ (US)

(73) Assignee: Predict Disease Private Limited, Ahmedabad (IN)

(21) Appl. No.: 16/210,885

(22) Filed: Dec. 5, 2018

Related U.S. Application Data
(60) Provisional application No. 62/595,394, filed on Dec. 6, 2017.

(51) Int. Cl. G16H 50/30; G16H 10/60; G16H 10/20

(52) U.S. Cl. CPC G16H 50/30; G16H 80/00; G16H 10/20; G16H 10/60

(57) ABSTRACT

The invention provides a system and method for identifying and predicting risk level of medical conditions of a patient comprising a web server for hosting and serving web pages to network connected nodes or devices upon request. The system also provides a database server connected to the web server for storing data related to one or more patients. The system further provides an application to access and process data from the database server and a visualization tool stored on the database server, the visualization tool configured to display graphics and data related to medical conditions of the one or more patients.

### CROSS-REFERENCE

This non-provisional application claims the benefit of U.S. provisional application No. 62/595,394 dated Dec. 6, 2017 and incorporates herein all the features filed with the provisional application.

### FIELD OF THE INVENTION

The present invention relates to a system and method for improving medical care by assisting and automating medical diagnosis and risk analysis which is made available to patients and healthcare professionals. The present invention pertains particularly to methods for gathering, analyzing, transforming and processing disparate data gathered from a plurality of sources to more accurately perform a patient risk analysis for medical conditions and illness.

### BACKGROUND OF THE INVENTION

Current methods available to a healthcare professional or patient to assess risk or diagnose an illness occurs by collecting and evaluating information about the patient, then determining what disease or condition best fits the information. The information gathered from the patient usually is processed to reach a diagnosis by using a protocol learned during the professional's education and training, which may be modified and updated by his or her medical experience. The protocol is an ordered process by which a healthcare professional ascertains information by examining the patient that allows the professional to rule out possible diseases until enough information is gathered to eliminate all but the diagnosed condition or risk factor.

One problem in the field of medicine is how to qualify patient provided information and data. Patient communication skills may be poor, or because of various reasons a patient may not be completely forthcoming when divulging habits and lifestyle. Another problem is that health care professionals may not be asking the right questions based on patient provided data. Additionally, health care professionals may not have access to a comprehensive knowledge base including the most recent medical trends and dynamic medical information required to accurately diagnose and assess a patent.

There is currently a need in the medical field for a system that provides an interface to both patients and health care professionals enabling data input from both, displaying probabilities and statistics processed from the input data as well as data gathered from historic medical information of the patient, the knowledge base and disparate data gathered from the patients presence and communications on associated Websites and groups such as chat rooms and social media.

### SUMMARY OF THE INVENTION

The present invention provides a system for identifying and predicting risk level of medical conditions of a patient comprising (i) a web server for hosting and serving web pages to network connected nodes or devices upon request; (ii) a database server connected to the web server for storing data related to one or more patients; (iii) an application to access and process data from the database server; and (iv) a visualization tool stored on the database server, the visualization tool configured to display graphics and data related to medical conditions of the one or more patients.

In an embodiment of the system, the patient data is received by the database server through (i) information provided by the patient on the web server; (ii) information provided by the physician on the web server; (iii) information gathered from the available research and literature data; and (iv) information scraped from public sources about the patient.

In another system embodiment, the database server includes a Natural Language Processing module to transform scraped and input data into medically usable data.

In one system embodiment, the Natural Language Processing module includes a computerised visual processing module for generating medical data using facial recognition, pattern recognition and item identification.

In a preferred system embodiment, the visualization tool provides a risk score and recommendation to the patient and the physician.

In a system embodiment, the database server further includes data from comparison groups for other patients and physicians stored in the database server.

The invention also provides a method for identifying and predicting risk level of medical conditions of a patient comprising (i) gathering information associated with the patient from one or more data sources; (ii) storing the information in a database server; (iii) processing the information from the database server; and (iv) displaying data and graphics related to medical conditions of the one or more patients on the visualization tool.

In a method embodiment, the processing of information is done by a Natural Language Processing module.

In a preferred method embodiment, the Natural Language Processing module can scrape and process information from publicly available data for patients and physicians.

In another method embodiment, the visualization tool displays one or more of a preliminary assessment report, risk assessment report, patient notes report, physician recommendation report and patient result consolidation report.

### BRIEF DESCRIPTION OF THE SEVERAL VIEWS OF THE DRAWINGS

FIG. 1 is an architectural diagram of a system 100 implementing Predict Disease according to an embodiment of the present invention.

FIG. 2 is one embodiment of a process flow describing data processing of the present system.

FIG. 3 is a screen shot of a gaming interface page 300 according to an embodiment of the present invention.

FIG. 4 is an embodiment of a system 400 using NLP to process scraped data.

FIGS. 5a and 5b, combined, comprise a process flow chart 500 describing a physician and patient interaction, according to one embodiment of the present invention.

FIG. 6 is a screen shot of a Physician Dashboard 600 according to an embodiment of the present invention.

FIG. 7 is a screen shot providing a Patient Dashboard, according to an embodiment of the present invention.

FIGS. 7a and 7b comprise a screen shot of a Patient Examination Screen 700 according to an embodiment of the present invention.

FIG. 8 is a screen shot of a stand-alone application showing an interface 800 for generating an Initial Risk Assessment Report according to an embodiment of the present invention.

FIG. 9 is a screen shot of an embodiment providing a Social Determinants of Health screen 900 according to one embodiment of the present invention.

FIG. 10 is a screen shot of a Data Scraping Screen 1000 which may be used to control data scraping as referenced in FIG. 3, Element 301, according to one embodiment of the present invention.

FIG. 11 is a screen shot of a Patient Comment Screen 1100, according to one embodiment of the present invention.

FIG. 12 is a screen shot of a Classification Screen 1200 shows a process for classifying keywords and matching known medical terms, according to one embodiment of the present invention.

FIG. 13 is a screen shot of a Physician Finding Screen 1300 which may be used by a physician to summarize and edit data for a patient, according to one embodiment of the present invention.

FIG. 14 is a screen shot of a Risk Assessment and Scores screen 1400, according to one embodiment of the present invention.

FIG. 15 is a screen shot of a Physician Risk Dashboard screen 1500, according to one embodiment of the present invention.

FIG. 16 is a screen shot of a Patient Result Consolidation screen 1600, according to one embodiment of the present invention.

FIG. 17 is a screen shot of a Symptoms Search Results screen 1700, according to one embodiment of the present invention.

FIG. 18 is a screen shot of a Physician Recommendation, according to one embodiment of the present invention.

### DETAILED DESCRIPTION OF THE INVENTION

The inventors provide a unique process and system that provides enhanced risk analysis and visualization for medical professionals and patients. The present invention is described in enabling detail using the following examples, which may describe more than one relevant embodiment falling within the scope of the present invention.

Predict Disease is an Internet and software based system coined by the present inventors. This system and method provides a medical condition prediction and recommendation system. It predicts the likelihood of patient getting diabetes and other conditions much in advance of the patient actually being diagnosed with a serious medical condition.

The main objective is to identify high-risk patients during regular body checkup/primary care and provide customized treatment to reduce the risk for delaying or preventing the condition. Diabetes and other chronic diseases display symptoms gradually over long periods of time and involve genetics and many lifestyle factors. Commonly people experience early signs and symptoms that are missed by healthcare professionals and patients, as well. Such signs and symptoms may not seem significant or life threatening and usually taken for granted by the patient or professional.

Health care professionals in the current medical climate are pressured by their business office models and insurance companies to be productive and efficient, seeing as many patients as possible in a given day causing doctors to rush through exams and hastily making assessments and diagnosis.

The Predict Diseases prediction system provide the solution for the above problems and assists the patient and health professional to identify the risk level of a patient well in advance of the patient actually developing a given medical condition. The system may utilize day-to-day patient information gathered from a plurality of sources to predict the risk of a medical condition and calculate risk score without a need to perform expensive lab tests or be referred to specialized physicians.

The system provides real-time visibility to physicians around similar patients with the same characteristics and similar symptoms, including diabetes and other conditions. By evaluating multitudes of medical data of similar type patients, the physician may be enabled to ask the right questions, and evaluate previously unidentified but related factors which helps lead the physician to identify how much a patient is at risk of developing a specific medical condition.

The system employs at least two versions, a stand-alone application for patients, which allow patients to input data and perform a self-assessment before visiting a doctor, and a physician or health professional application that allows further examination of a patient and calculate risk score based on additional findings from gathered data and access to a dynamic knowledge base.

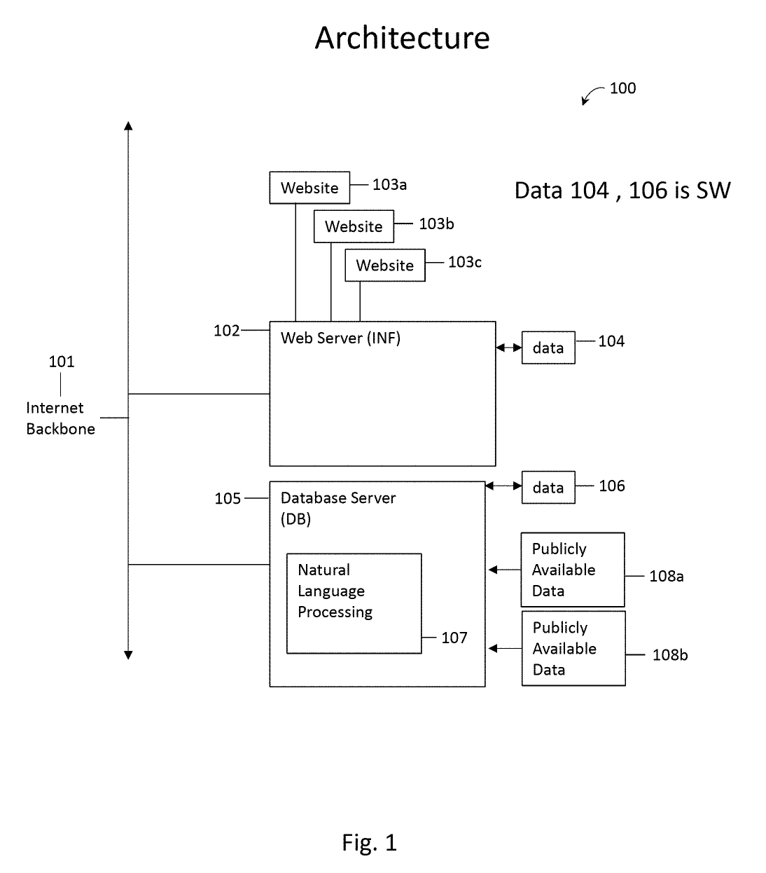

FIG. 1 is an architectural diagram of a network 100. The network 100 includes the well-known Internet network depicted herein by an Internet backbone 101. Backbone 101 represents much of the lines, equipment, and connection points that make up the system as a whole including any connected sub-networks. Therefore, there is no geographic limitation to the practice of the present invention.

Internet backbone 101 supports an information (INF) server 102 adapted as a Web server for hosting and serving web pages to network connected nodes or devices upon request. Web server 102 includes access to at least one data repository 103 for holding data regarding subscribers of the service including patient and provider information, profile data of subscribers, account status, patient questionnaires, etc. Virtually all data collected and input in the system is stored in data repository 103.

Software 104 operates in concert with Web server 102 and gathers data scraped from publically available websites 103a, 103b and 103c, news feeds, articles and current medical research publications relating to current medical information and trends. This may be done automatically by the software navigating to various sources in order to identify and scrape specific data. This data may be stored, analyzed and processed then provided to database server 105 or served directly to network connected nodes or devices upon request by a patient or medical professional.

Database server 105 stores data regarding subscribers of the system including medical history, professional users, information directly input by the subscribers, information from scraping the Internet, as well as a comprehensive dynamic medical knowledgebase. In one embodiment, database server 105 may host a software application 106 (SW) which may directly access data to store for later processing and update the knowledgebase. Software 106 also may serve to process raw data, data from Natural Language Processing (NLP) 107 and additional information into graphic and readily understood statistics served to in interfaces at a patient and health professional.

The knowledgebase may contain all known medical data available from virtually any publically available source, including text books, medical journals, articles, physician and patient provided data and even social media platforms. The knowledgebase may also automatically update its own information when conflicting data is gathered that may contradict what the system has previously stored or understands.

Natural Language Processing (NLP) 107 may be implemented as part of database server 105 or be a standalone component. NLP 107 transforms scraped and input data into medical related data in useable formats before storing in server 105. In another embodiment, database server 104 may store input data unchanged, and NLP 107 processes raw data just prior to processing by SW 106.

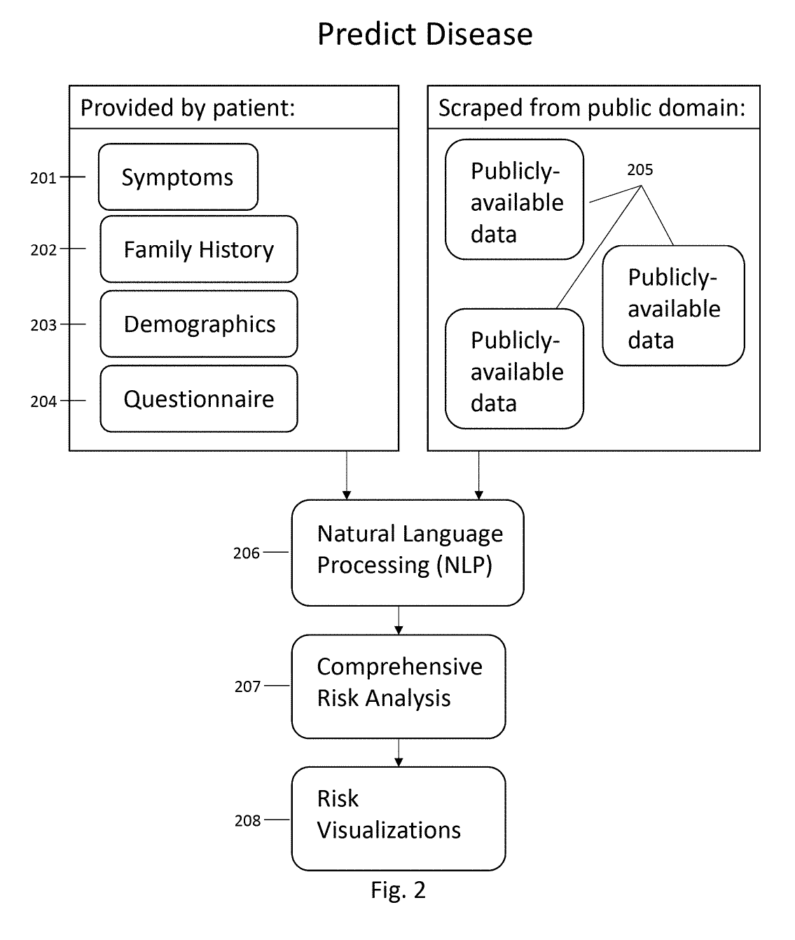

FIG. 2 is one embodiment of a process flow describing data processing of the present system. Data for each patient may be collected from one or more sources, possibly including but not limited to: current symptoms (201), family history (202), and demographics (203). A patient may also provide information in written form, such as filling out a questionnaire (204). Data may also be scraped from public sources (205) automatically. For example a patient may be a member of various social media platforms and have subscriptions to commercial websites indicating travel plans, financial status and shopping sites. The patient subscriber may provide Web server 102 URLs and login credentials enabling software 104 to automatically gather personal information that may have a bearing on the overall health of the patient. If necessary, data is interpreted using Natural Language Processing (206). The data is used to create reports of risk analysis (207) and visualizations of patient risk profiles (208).

NLP 107 is enhanced to identify and correct medical information provided by the patient, such as slang and spelling mistakes input in a questionnaire or scraped from the Websites 103a-103c. Additionally, the NLP 206 may correct terms identified differently based on race and culture of the patient. NLP 206 may be implemented with image detection software identifying images that may be related to the health of a patient, for example, images showing the patient at parties, drinking alcohol, and hiking. The NLP may then generate data based on the images that is input in the data processing prior to visual display of results in the interfaces of patients and health professionals.

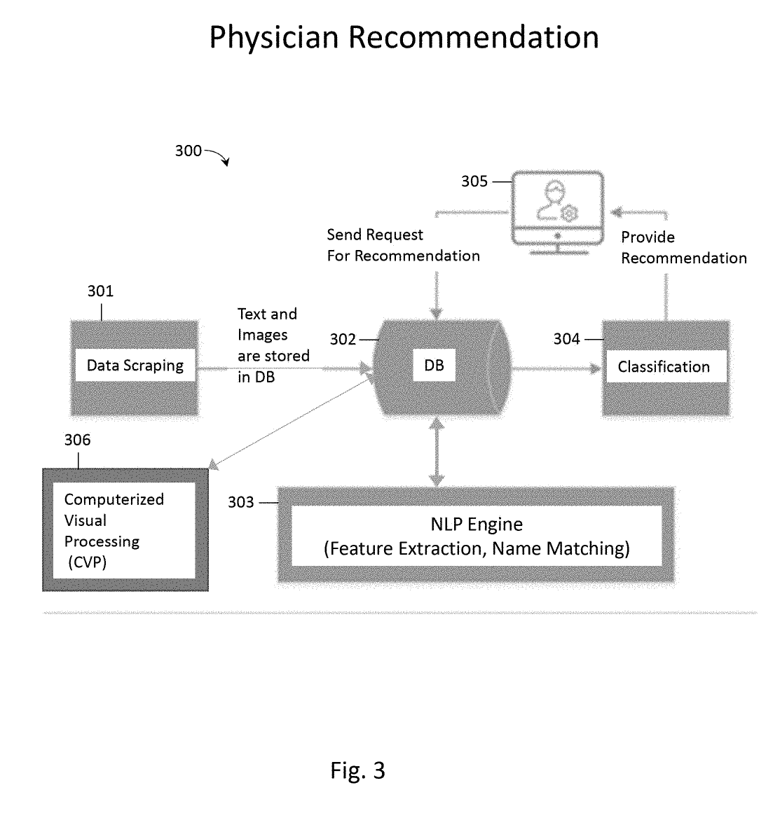

FIG. 3 is a process flow chart showing a system 300 for collecting input data from a patient and transforming and classifying it to provide a user with a recommendation, according to one embodiment of the invention. Using login credentials provided by a physician, data scraping 301 is a software program such as a bot or a web crawler that logs in to the patient's various accounts, as if the patient with the login credentials provided by the patient when registering with the system. The bot or web crawler navigates to Websites and interfaces having personal information regarding the patient and scrapes text and images from available data. The scraped data is stored in DB 302 similar to Database 102 of FIG. 1 along with a medical database that is also known as a medical knowledge base.

The NLP Engine 303 corrects spelling and grammar mistakes, translates extra information such as “Internet slang” and emojis, and identifies key words and matches to known medical terms. NLP Engine 303 then assigns an accurate plain meaning to the corrected terms. The NLP passes the corrected terminology back to DB 302 for future analysis.

Computerized Visual Processing (CVP) is part of the enhanced NLP interpreting image data along with associated text and metadata from the scraped data stored in the DB. Using facial recognition, pattern recognition, and item identification, CVP generates relevant medical data from images, text and metadata and passes it back to DB 302 for later analysis. This data may then be analyzed, normalized and corrected, for instance using a Computerized Visual Processing screen as shown in FIG. 3. In one embodiment facial recognition is implemented to identify patients in specific settings in an image, for example hiking or at a party, or holding an alcoholic beverage or even eating a specific item. With the incorporation of metadata associated with scraped images, and functions of the NLP, medical data for a specific patient, or relatives and friends of the patient can be rendered, interpreted and stored in the DB for use in making a risk recommendation and serving patient data to a health professional.

Later, a user such as a health care professional sends a request for a recommendation 305 for a patient. Classification 304 is software part of DB 302 or operating at an accessible server that classifies the stored data into categories relevant to medical data stored in the knowledgebase. The combined software analyzes stored, processed data from the NLP, including the CVP rendered data along with input from the patient questionnaire, and identifies a specific medical condition along with a quantification of risk for said condition. A risk analysis recommendation may then be made available to a health professional prior to or while conducting a physical evaluation of the user patient.

With this information, a health professional may have a better base and understanding of a current state of the user patient. The health professional may then focus a physical examination including further tests to further evaluate and treat the patient. The data may also be made available to the user patient in order for the user patient to better understand their own state of health.

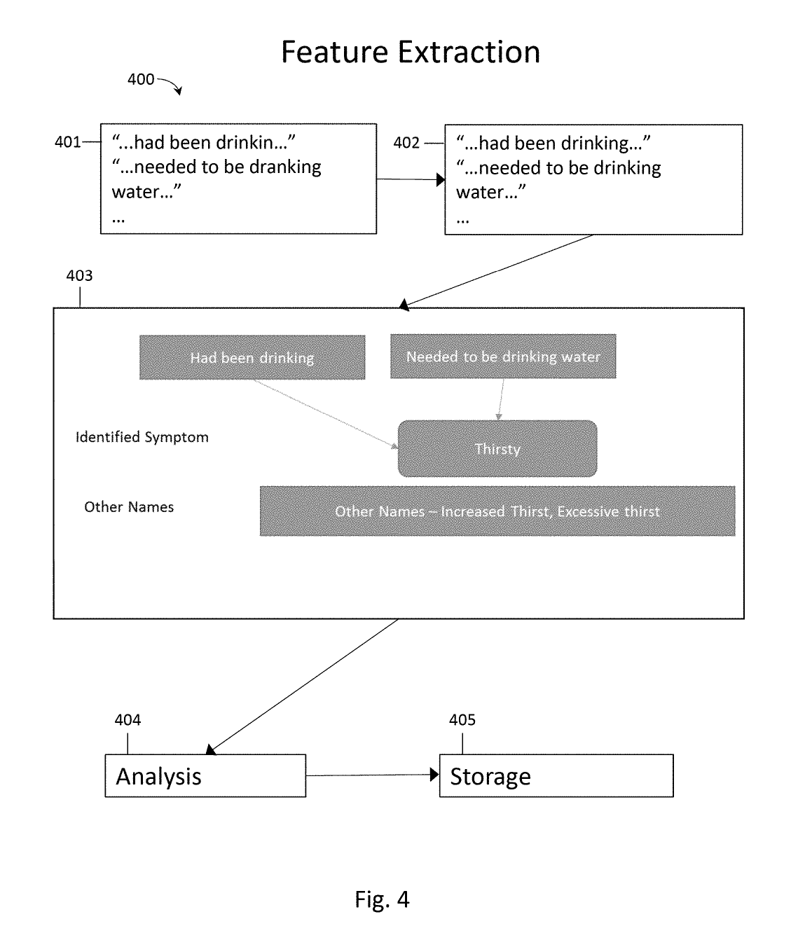

FIG. 4 is an embodiment of a system 400 using NLP to process scraped data, identify keywords and store them for future use. Text data, which may contain grammatical errors and/or spelling errors, is input as shown in step 401. The input data is corrected for grammar and spelling as shown in step 402. The corrected data is processed using an extraction algorithm as shown in step 403 to identify key words and matches to known medical terms. These terms are classified as shown in step 404. This may be done using software such as the Classification software mentioned in FIG. 3, Element 304. The classification results are stored in a database, such as the DB mentioned in FIG. 3, Element 302. In another embodiment, a knowledge worker may assist in normalizing language input into the system.

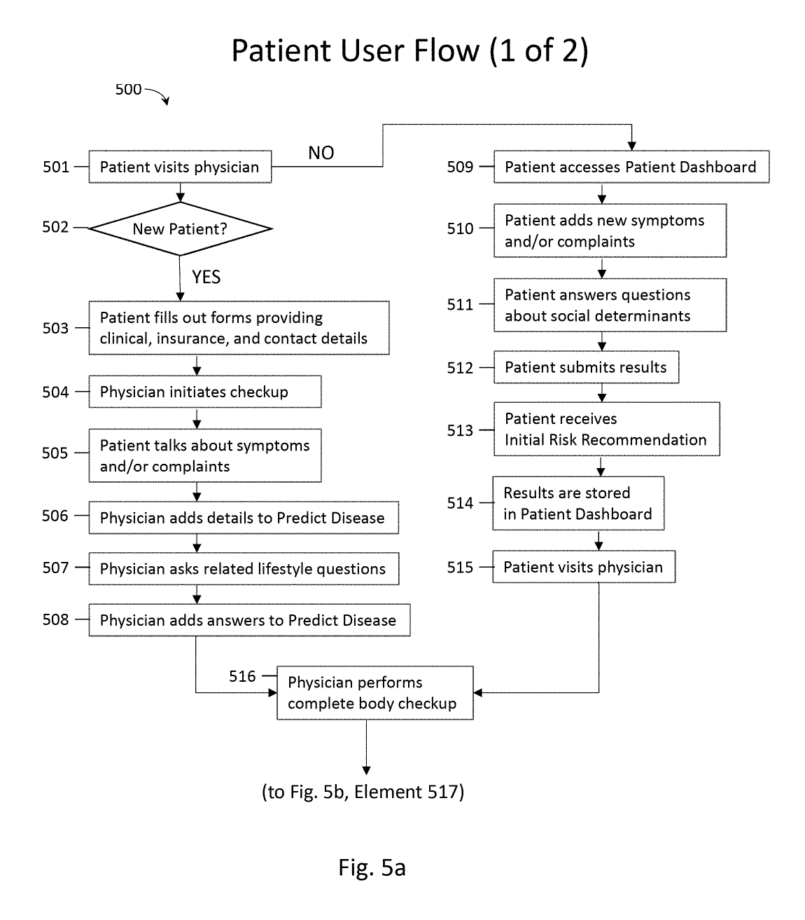

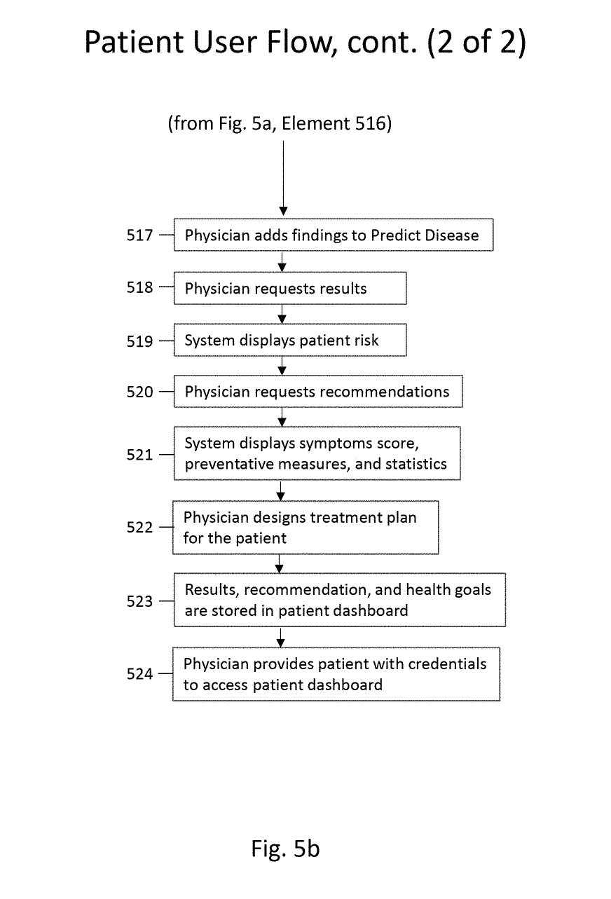

FIGS. 5a and 5b, combined, comprise a process flow chart 500 describing a physician and patient interaction, according to one embodiment of the present invention. A patient visits a physician as shown in step 501. The physician checks the system in step 502 to determine if the patient is a new patient or an existing patient. If the patient is a new patient, the patient fills out forms with medical questionnaire, insurance, and contact details in step 503. Then, a physician initiates a checkup at step 504. The patient describes symptoms and complaints to the physician in step 505 and the physician adds this data to an input interface of Predict Disease in step 506. The physician asks related social determinants of health questions in step 507 and adds this data to Predict Disease system in step 508.

In an alternative embodiment, if the patient is an existing patient, the patient accesses a Patient Dashboard in step 509, which is an interface where the patient views and provides additional information. The patient uses the Patient Dashboard to add information such as new symptoms and complaints as shown in step 510. The patient answers questions about social determinants, such as ethnic background and geographic location, in step 511 and submits the answers to Predict Disease in step 512. Predict Disease then processes all of the information as described in FIG. 3 and the patient receives a risk recommendation at step 513 which is stored in the DB accessible at the Patient Dashboard in step 514. The patient then visits the physician in step 515.

At this point, for new and existing patients, the physician performs a complete physical examination in step 516 and adds the results of the examination to Predict Disease in step 517. The physician requests to calculate the risk in step 518. The physician requests the Risk Recommendations in step 520 and Predict Disease provides the patient's Risk score, possible preventative measures, and statistics in step 521. The physician may design a treatment plan for the patient in step 522. Predict Disease stores the results of the risk assessment, the patient recommendations, and patient health goals in patient dashboard as shown in step 523. The physician may give the patient the necessary credentials to access the Patient Dashboard as shown in step 524.

In another embodiment, a patient accesses a Patient Dashboard without the aid of a physician to receive a Risk Recommendation. The patient logs in to a Patient Dashboard using secure credentials to ensure patient privacy. Using the Patient Dashboard, the patient may perform one or more of the following tasks: the patient may update personal information such as contact information and insurance, the patient may update family history and demographics, the patient may answer questions such as filling out a questionnaire, the patient may update symptoms and past or present diagnoses, and the patient may submit comments about how they are currently feeling. In one embodiment, the patient may provide login credentials to allow data scraping from public websites which may contain information relevant to the patient. In another embodiment, the patient may provide additional information by authenticating through other websites using a Social Login. Predict Disease system then processes all the information as described in FIG. 3 and the patient receives a Risk Recommendation.

In another embodiment, a patient accesses a patient dashboard without the aid of a physician to follow up on a Risk Recommendation. A patient logs in to a Patient Dashboard using secure credentials to ensure patient privacy. Using the Patient Dashboard, the patient may perform one or more of the following tasks: the patient may update personal information such as contact details and insurance, the patient may input new symptoms and complaints, the patient may input biometric data such as weight and water consumed per day, the patient may request visualizations of available data including patient data and risk scores, the patient may request a Risk Recommendation which may include a recommendation about visiting a physician and if so, may recommend which type of physician to visit. The Risk Recommendation may include suggestions to lower patient health risks in the future. The Risk Recommendation may include health goals customized to the patient.

Notes regarding the patient may be provided in a physician examination screen of Predict Disease. In this embodiment, every time the patient comes to see a physician for examination, the physician usually writes notes. NLP algorithms may identify symptoms in real time and show it to doctor what information has been identified. After the physician validates then clicks on submit, this new information will go to database. The note will go to the database, but during prediction, it may not be used because not all patients have chronic diseases so the physician will have to eventually check mark so that application can understand which comment to use in prediction.

The application may ask social determinants of health questions not commonly added to medical data. This information may include lifestyle, sleep conditions, food habits, financial situation, and sleep cycle. The application reviews the responses on each question and calculates the risk score for this category. Imbalance in such areas increases the risk of diabetes. As per the research, lifestyle factors play more important role in increased risk of diabetes then clinical factors. Therefore, this category is made more intuitive by adding one more feature that is called “Share your feelings”. What it does is ask user/patient/person to type his/her feelings in simple English, and then, our system read the opinions and measure the sentiments of a person and categorize if a person is happy, sad, angry, stressed, emotional and also identify the cause of the negative feelings. The reason this feature is provided is because no questionnaire can be sufficient to capture a person's feelings other than the person himself or herself.

The type of information gathered from and on behalf of the patient as well as other data gathered from physician, social media and other data sources is to fundamentally understand a 360-degree view of the patient, in all aspects of their lives, not just restricted to what is understood as medical data. Understand how the patient behaves, act, clinical aspects, lifestyle aspects, understand communication so that better care can be provided.

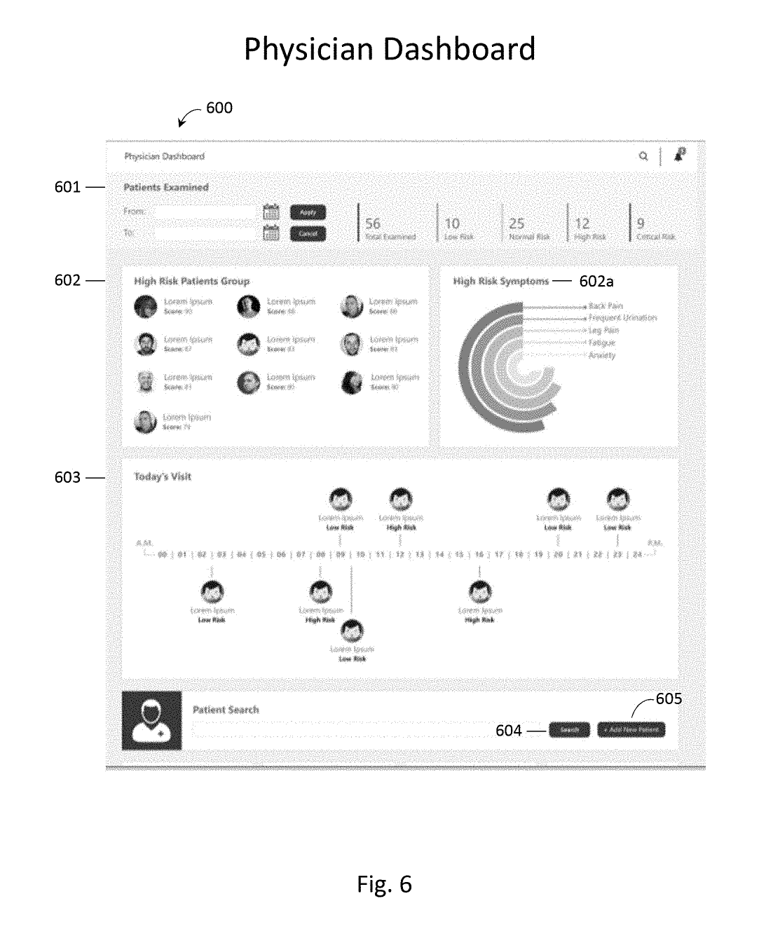

FIG. 6 is a screen shot of a Physician Dashboard 600 according to an embodiment of the present invention. Search fields 601 allow a physician input search queries for name or date, as in this example, to narrow the results of the patient data they are viewing in the Physician Risk Dashboard. Section 602 is a High-Risk Patients Group showing the highest risk patients to the physician in text and picture format. In this example, the medical condition is diabetes. 602a shows a graphic of symptoms expressed by all of the high-risk patients for diabetes which gives the physician a quick graphic of what symptoms to look for in other patents being analyzed for high risk. Section 603 shows Today's Visit which provides a detailed chronology of each patient's interactions for a given timeframe. In this embodiment, the physician may drill down to patient data by interacting with each patient icon. Patient Search button 604 allows a physician to search for an existing patient. Add New Patient button 605 allows a physician to add a new patient data to the system.

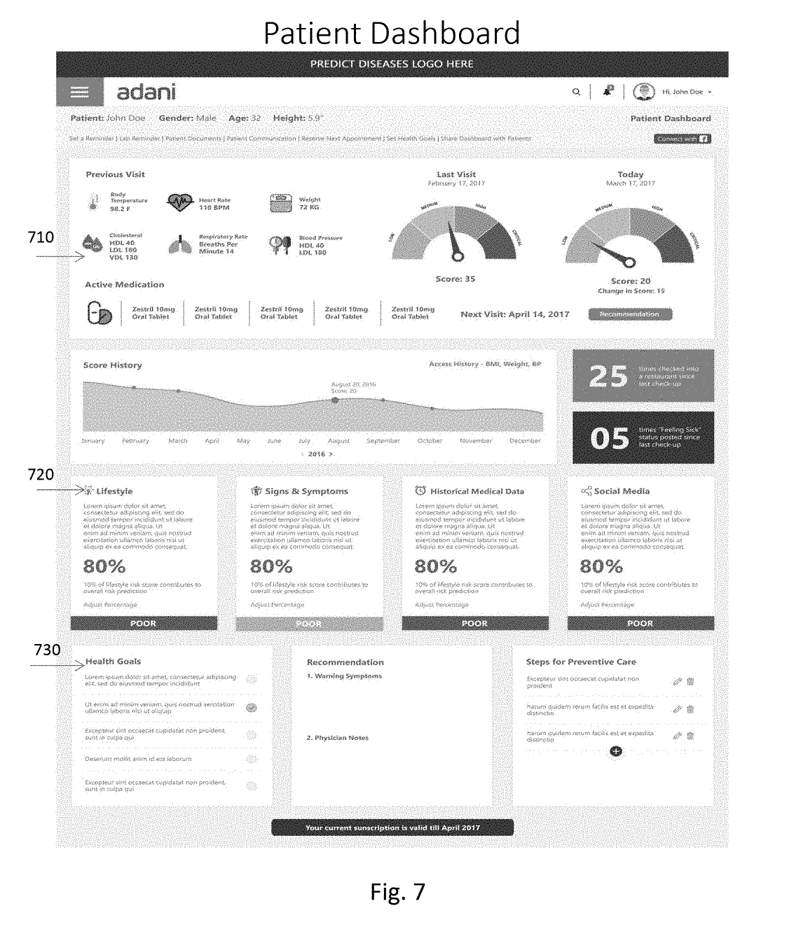

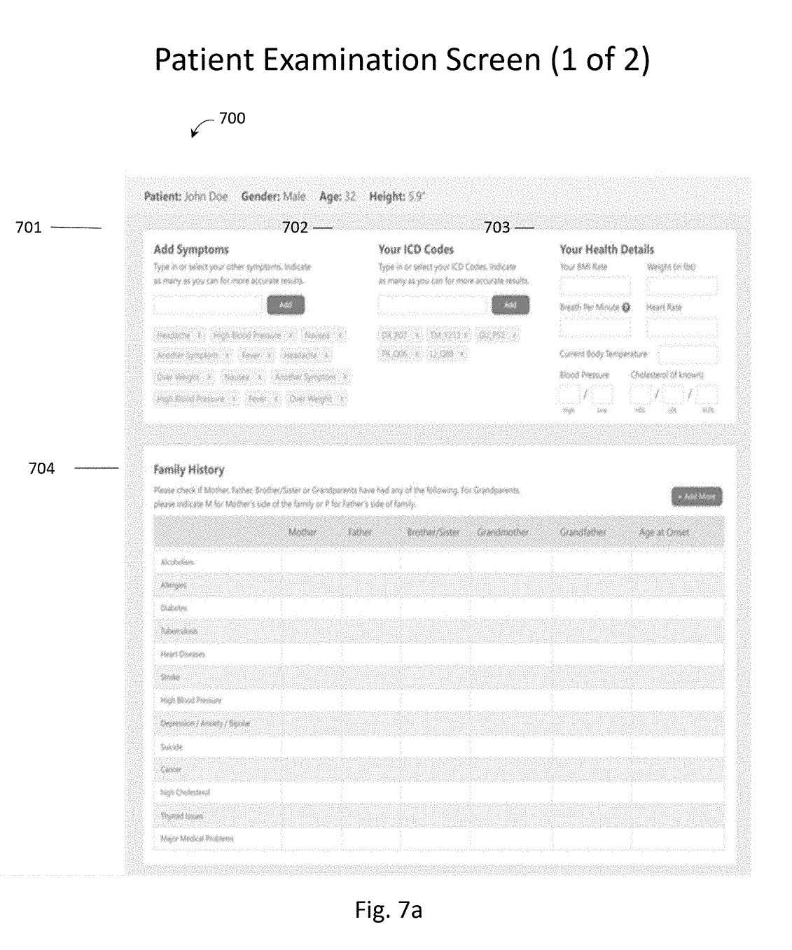

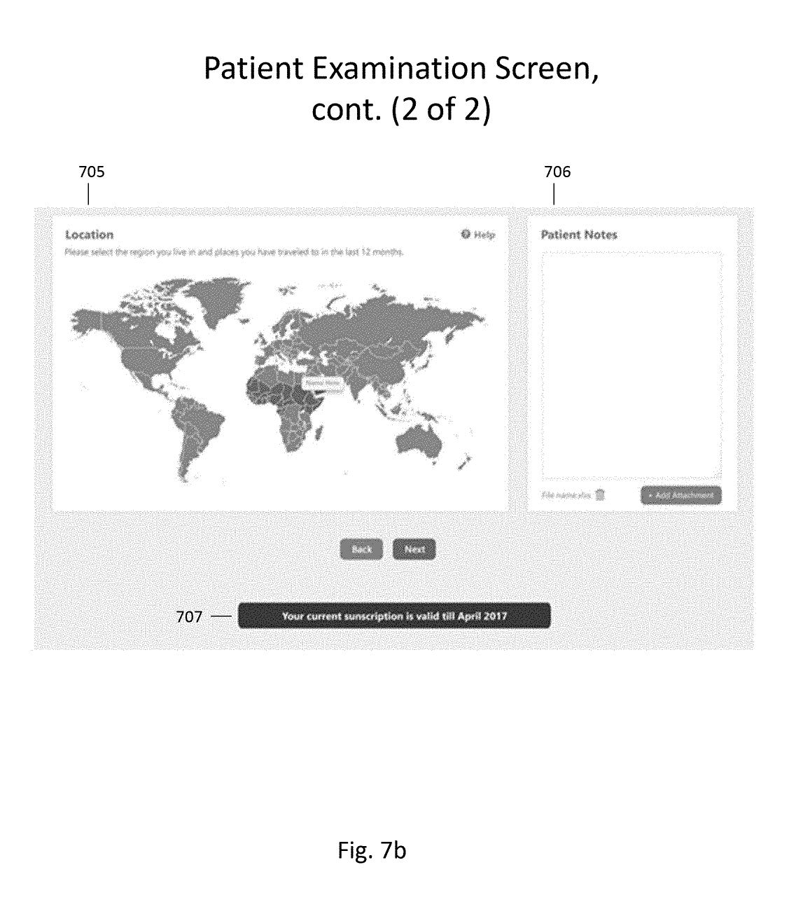

FIG. 7 is a screen shot of a Patient Dashboard. The patient dashboard enables a 360-degree view of the patient providing data related to present and historical medical data, social and clinical metrics. The patient dashboard is a screen providing a visual representation of data stored in the database regarding historical risk score, other details about the patient and shows the pattern of progress to the physician or/and patient. Every time patient uses the standalone application for self-risk assessment or visits to the doctor for the checkup, the risk score and recommendations (from recommendation page) get stored in the patient dashboard.

The patient will have access to patient dashboard so the patient can visit the dashboard to perform tasks, such as create a self-assessment in a stand-alone implementation; or the doctor may access the patient dashboard for viewing patient information, examining risk scores and statistics as well as adding information regarding the patient prior to or during a medical examination. In this embodiment at 710, the patient's medical history comparisons are provided for various dates, scores are assigned for improvements or degenerations and a list of current medications are shown. A score history graph depicts weather the patient scores are improving or declining.

Scores are also provided at 720 indicating a relationship between lifestyle, signs and symptoms, historical medical data, social media and risk factor for a particular disease, shown as a percentage. 730 is an area on the patient dashboard indicating fields for a physician to enter data. Here the physician may indicate when a health goal is achieved, provide steps for preventative care and begin an examination process.

The invention also provides risk scores for a patient's susceptibility to or chance of eventually developing a certain disease relative to available research and literature. Every year there are millions of research papers and literature published around diabetes type 2, for example. In order to publish the research paper, the research organization conducts extensive research on various types of data related to a particular disease and then publishes their research outcomes as data. Therefore, all available research outcomes are leveraged, in one embodiment, to calculate the risk score. The research and literature data is scraped from publically available data, and stored in the knowledgebase or database.

Developing the risk score from these additional sources would be more accurate because there has been extensive research conducted to come up with certain findings or statistics regarding a particular disease, thereby implementing identified risk metrics as part of research outcome.

For example, Risk for developing type 2 diabetes varies somewhat among different ethnic and age groups. According to one recent national survey the prevalence of type 2 diabetes (what percentage of people had the disease at a given point in time) was 1:
7.1% among whites (non-Hispanic)
8.4% among Asian Americans
11.8% among Hispanics
12.6% among African Americans
   Additionally, the prevalence among certain Native American groups was as high as 33.5%. Statins Increase Diabetes Risk by up to 50% in Older Women. If both parents have diabetes type 2, then risk for developing increases by 30%. If distance relative have diabetes type 2, then risk for developing increases by 5%. One with skill can understand using such statistics would give a better prediction than just using the clinical records.

In a method of this embodiment the risk score would be calculated based on any of a patient's demographics. Clinical and external factors are matched with the risk metrics. It could go up or down based on the patient's characteristics (demographics, clinical or external factors).

For example, a risk score may be developed as follows:

### Risk Metric—Statins Increase Diabetes Risk by up to 50% in Older Women.

Risk Metric—Exercise could reduce the type two diabetes by 25% for women between age of 30 and 65.

### Target Patient

### Age—56

### Gender—Female

### Symptoms—Frequent Urination, Backpain

### Medication—Statins

Physician's note—Patient gets extremely tired after lunch time and have been increasing the weight since last 2 months. Patient mentioned that she has been regularly exercising for at least 1 hour everyday.

Based on above information, the system will start calculating risk score for all methods that has been explained above. For this method, the system will calculate the risk as shown below.

Available metrics based on patient characteristics:

### Risk Metric—Statins Increase Diabetes Risk by up to 50% in Older Women (55+)

### Age=56 (Present, 55+)

### Gender=Female (Present, Women)

### Statins=Present

### Risk Threshold=+Max 50%

Final Risk=+35% (for example, risk score will be calculated taking all characteristics into consideration)
Risk Metric—Exercise could reduce the type two diabetes by 25% for women between age of 30 and 65

### Age=56 (Present, 55+)

### Gender=Female (Present, Women)

Exercise=Present (1 hour+)

### Risk Threshold=(−) Max 25%

Final Risk=−20% (for example, risk score will be calculated taking all characteristics into consideration) Risk score based on research metrics=Risk Metrics 1+Risk metrics 2=+35+(−20)=15%. Physicians will have ability to upload research paper and other literature around diabetes type 2 and then, the system will automatically update the pool of risk metrics.

The feature allows the target patient to be compared with any similar diabetic patient to measure the risk. The system will identify a list of diabetic patients based on the target patient demographics and clinical details. Then, target patient can either select from the pre-defined list or create a new comparison group and create patients for the comparison group. The advantage is that person/target patients can regular compare him/herself with diabetic closed relatives or friends who share similar demographics and/or hobbies, activities and preferences. The feature will be available to physician and patient standalone application.

FIGS. 7a and 7b comprise a screen shot of a Patient Examination Screen 700 according to an embodiment of the present invention, as referred to in FIG. 5, Element 509. Symptoms panel 701 enables a patient to enter symptoms they have experienced. ICD Codes panel 702 is where a patient may enter ICD Codes. Your Health Details panel 703 is where a patient may enter current details regarding physical condition and health. Family History panel 704 enables a patient to enter information relating to family members, their demographics and their known diagnosed medical conditions.

In an embodiment, and for example, research studies suggest that the lifetime risk of developing T2D is 40% for individuals who have one parent with T2D and almost 70% if both parents are affected. In fact, not only parents but other close relatives such as, brother, sister, uncle, aunt, grandparents, with diabetes can also impact the risk of developing the type 2 diabetes. It is therefore extremely important to analyze the risk based on family history.

Presently, all the web-based risk assessment tools calculate the risk of developing diabetes based on parental history and that is also based upon the answers on closed-ended questions i.e. yes or no. If a person answer “yes” for the question such as “do you have father or mother with diabetes?” Then, their system automatically adds the certain amount of risk to the final result. That is not an intuitive way of deciding the influence of family history on the overall risk score. The available solutions do not take environmental factors, and characteristics of a diabetic family member into consideration. We therefore, we have invented a unique methodology where the risk score will be calculated based on quantitative comparison between non-diabetic person and diabetic family members.

In the present invention, the system will compare and match the factors such as the demographics like age, weight, gender, height), environmental factors (stress, eating habits, job condition, income level, etc.), clinical factors (BMI, cholesterol level, other health condition). The algorithms used in the present invention for comparing and matching the various factors provides more accurate risk score than the closed-ended question/answer used in the available solutions in the market. The methodology used in the present invention helps in identifying whether the person is in a high, medium or low risk group based on a comparison with the family member's history based on current demographic, environmental and clinical factors.

The available solutions in the market, will tell you that you are in a risk of getting diabetes but that will not tell you when you should be worried and when should the necessary steps be taken by the patient.

Below is provided a scenario by way of an example for better understanding of the solution provided by the present system:

Father has Type 2 Diabetes

Onset Age (when it diagnosed)—34

Onset Weight—170 LB

Onset BMI—32

Onset Obesity Condition—Present

Onset Stress—High

Onset Other Condition—High Cholesterol

Non-Diabetic Person

Current Age—24

Current Weight—156

Current BMI—30

Current Obesity Condition—Present

Current Stress—Low/Not working

Current other condition—N/A

The present system will compare non-diabetic person profile with the diabetic father to calculate the current risk level. Based on the BMI, Weight and Obesity condition, system identifies that the person is in high risk level. In order to prevent or lower down the risk of diabetes, person should reduce the weight and do exercise. Such analysis and recommendation allows doctor to communicate patient more effectively and design the right treatment plan for patient. The present system is created to show a patient's risk level based on the current situation and to help the physicians to understand when the patient is facing a particular risk and needs to take preventive steps to reduce that particular risk. In an embodiment, the family history risk score will be added to the overall risk score of the patient.

In an embodiment, Location panel 705 allows a patient to enter locations they have visited recently and in the past. Patient Notes panel 706 allows a patient to enter any additional notes that they would like to add for their own benefit or for a physician. The patient note functionality allows patients to explain their complaints and/or health concerns in non-medical terminology. The present system will understand the text, analyze and convert into medical data for leveraging to calculate risk and generate recommendations. The patient note functionality helps patients to disclose everything in simple English and does not require the use of complex medical terms by the patient. The system then analyses the text provided in the patient note functionality and identifies the symptoms from the text to build the patient profile for patient risk assessment. In one embodiment of the present invention, the information entered in the patient examination screen will be stored, processed and made accessible to the physician. Additionally, in another embodiment, the patient adds information regarding Websites including social sites, networks, financial information, etc. which may be passed to the data scraping feature as referenced in FIG. 3, Element 301. The scraped data may be saved in the DB for future processing by the physician. In another embodiment of this invention, patient notes will only be surfaced to the physician. Subscription Status panel 707 shows the status of the patient's current subscription.

In one embodiment of this invention, a user could be allowed to delete or edit comments after they have been submitted. In another embodiment of this invention, a user would not be allowed to alter comments once they are submitted.

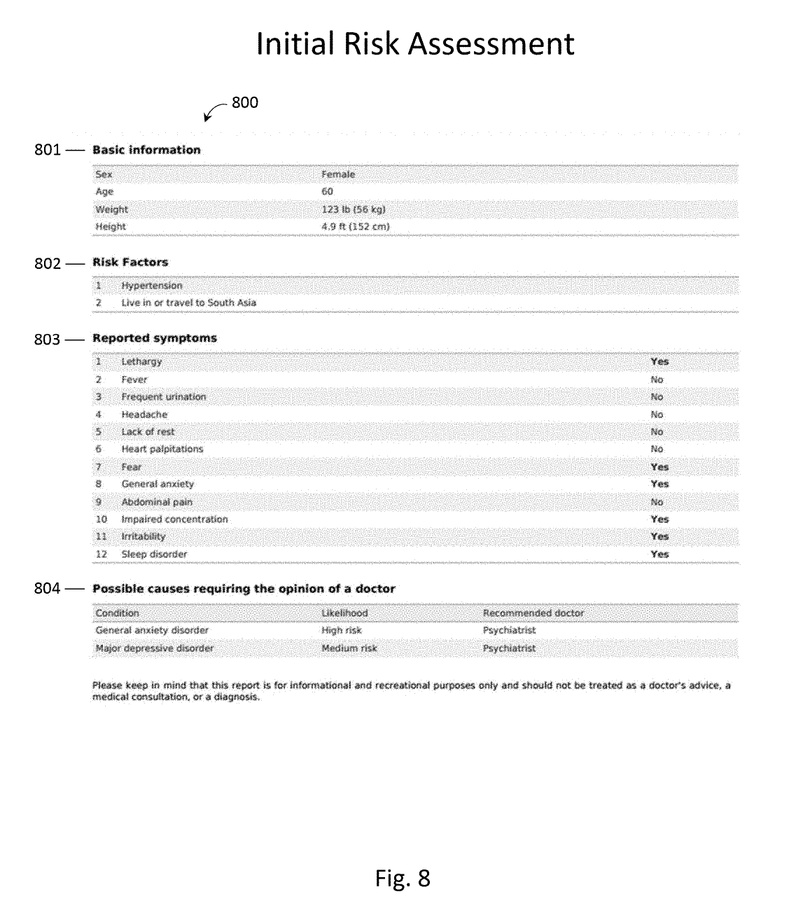

FIG. 8 is a screen shot of a stand-alone application showing an interface Initial Risk Assessment 800 according to an embodiment of the present invention. The information entered at this interface may be used to generate a Preliminary risk assessment report. Basic Information panel 801 shows basic patient information. Risk Factors panel 802 shows indicated risk factors based on data entered. Reported Symptoms panel 803 lists symptoms the patient has reported experiencing. Possible Causes panel 804 lists possible medical conditions, their likelihood, and the recommended doctor to consult for further information. In another embodiment of the invention, an interface for Initial Risk Assessment 800 is contained on a database server such as referenced in FIG. 1, Element 105 and has software to provide the interface to a user, such as referenced in FIG. 1, Element 106.

In an alternative embodiment, a patient may add credentials to a login page into the system. The system may then route the patient to the patient dashboard of FIG. 7a. The patient selects a Self-assessment option. The system then routes the patient to Patient examination screen at FIG. 8. After the patient provides symptoms, information and answers questions a Self-assessment report is generated and stored under Patient dashboard. This report may then be forwarded to the patient's physician.

The application provides access to patients for generating a self-assessment report outside of physician's office or before visiting doctor. The application will provide access to a “patient examination screen” and a “Social determinants of health questionnaire” to collect necessary data to generate the preliminary assessment report. If the Patient doesn't use the stand-alone application option and goes directly to the doctor for the checkup, then, obviously, the self-assessment report is not going to be generated. The purpose of self-assessment report to provide data before visiting physician.

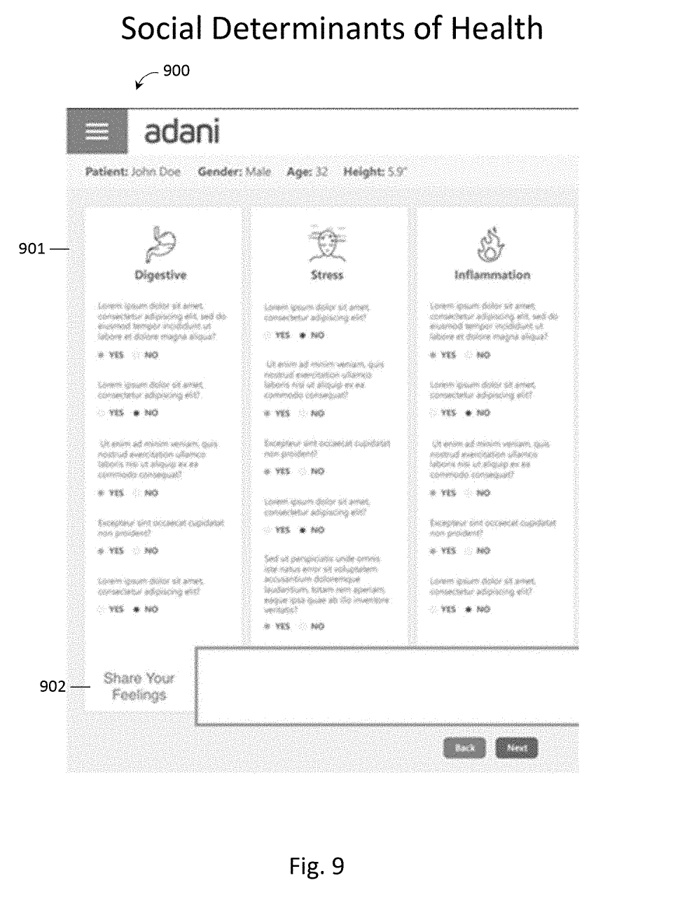

FIG. 9 is a screenshot of an embodiment providing a Social Determinants of Health Screen 900 according to one embodiment of the present invention. The Social Determinants of Health Screen includes a Questions Panel 901 which allows a patient to answer specific questions regarding their lifestyle, sleep conditions, food habits, financial solution, sleep cycle etc. The system reviews the responses to each question provided by the patient and calculates the risk score for this category. Research suggests that imbalances in the Social Determinants of Health factors increases the risk of diseases such as diabetes. For example, lifestyle factors play more important role in increased risk of diabetes than clinical factors. In order to make the analyses more effective, the invention also adds a feature “Share Your Feelings 902” in the Social Determinants of Health Screen. Share Your Feelings panel 902 invites a patient to describe their current physical state in a freeform written format. The feature requests the user/patient to provide his/her feelings in simple English and the system then analysis the feelings/opinions of the user/person to categorize whether the person is feeling happy, sad, angry, stressed, emotional, and thereafter the system identifies the cause of the feelings of the person. In one embodiment of the present invention, the written data will be passed to the data scraping feature as referenced in FIG. 3, Element 301. In this embodiment, the NLP will be implemented for any patient added text. In another embodiment of the invention, an interface for Social Determinants of Health Screen 900 is contained on a database server such as referenced in FIG. 1, Element 105 and has software to provide the interface to a user, such as referenced in FIG. 1, Element 106.

The Preliminary assessment report shows what the chances of the patient getting any chronic diseases based on symptoms and questionnaire along with risk score are. Every patient will be provided access to the system where the patient can go into the system and perform the self-assessment and generate the report. It's not mandatory, but it would help patients to keep track of their own risk factors outside of their physician's office. One of the objectives of the preliminary assessment report is to encourage patients to keep adding daily symptoms, external factors (lifestyle, stress, etc.) and complaints in the system. The resulting report would be forwarded to the doctor as well, and then software algorithms of the system will add examination's result from last doctor's visit with new symptoms that patient provides in preliminary assessment to monitor if the patient risk for a specific illness or disease is increasing. If it is determined that the risk is increasing, then the alert goes to a physician and then the physician may schedule an earlier appointment for repeated assessment.

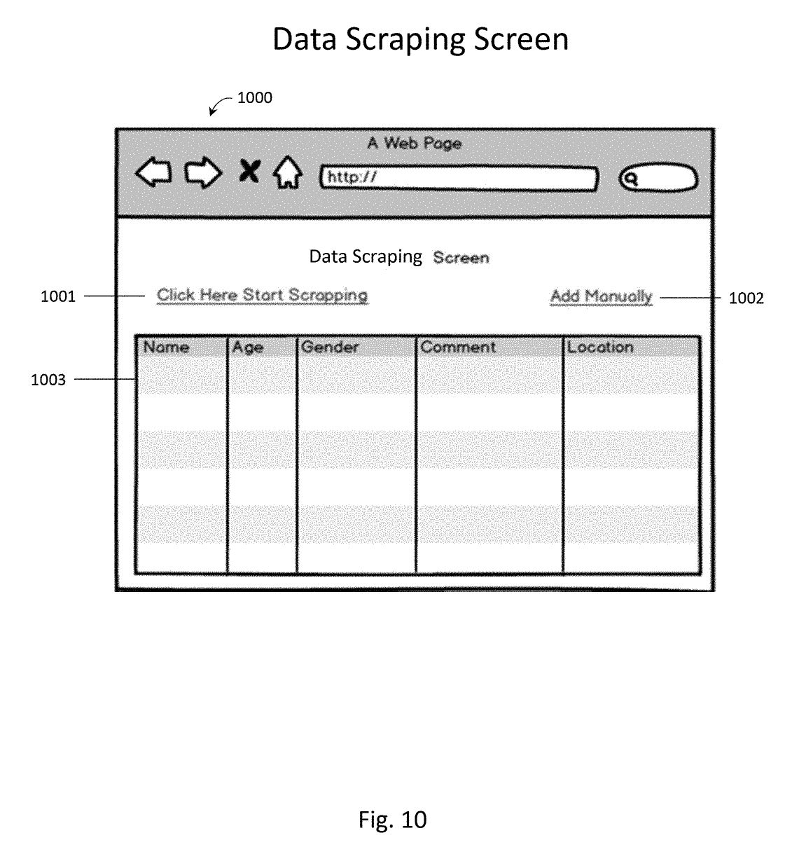

FIG. 10 is a screen shot of a Data Scraping Screen 1000 which may be used to control data scraping from multiple sources, as referenced in FIG. 3, Element 301, according to one embodiment of the present invention. A patient or physician may navigate to the web page of interest, wherein the address shows as an http address. Start Scraping button 1001 may be pressed to manually initiate a scrape of data for a patient at the selected Website. Alternatively, the software provides a bot or web crawler that may be implemented to automatically navigate to, and login as the user to a specific Website having patient personal data. Add Manually button 1002 may be pressed to provide the user with an interface to manually enter input data for scraping. A list field 1003 shows previously scraped data for patients.

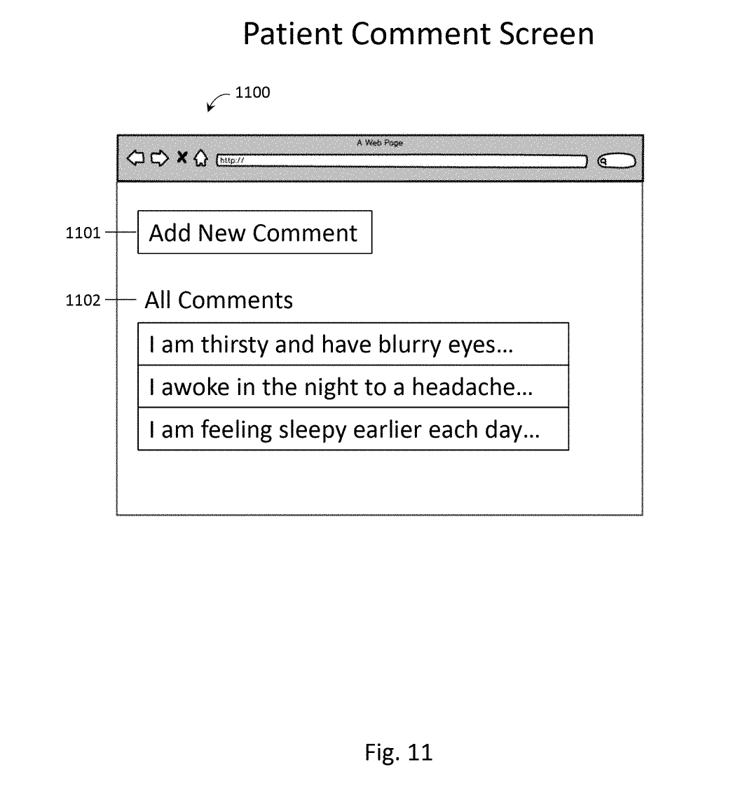

FIG. 11 is a screen shot of a Patient Notes Screen 1100 which may be used to enter patient notes as referenced in FIG. 7, Element 706, according to one embodiment of the present invention. Add New Comment button 1101 prompts the user for a new comment. All Comments button 1102 lists the entered comments for a given user. In one embodiment of this invention, a user could be allowed to delete or edit comments after they have been submitted. In another embodiment of this invention, a user would not be allowed to alter comments once they are submitted. Comments are stored in a database or DB, such as Element 302 in FIG. 3. In one embodiment, Predict Disease processes all of the comments as described in FIG. 3, using NLP to identify key words and matching medical terms. In one embodiment, a Risk Recommendation, for example as shown in FIG. 5, Element 513, contains all of the comments. In another embodiment, a Risk Recommendation, for example as shown in FIG. 5, Element 513, contains all of the comments and also contains key words and medical terms discovered by the NLP Engine, for example as shown in FIG. 3, Element 303.

The primary objective of implementing the NLP into the system is to understand the natural language and then identify vital information from the text. For NLP to understand the text, you have to train the NLP engine to recognize the future text by adding lots of similar data (such as wordings, sentences, spellings, etc.) into the database. However, there is a major possibility that not all presented text can be identified by the system because you never know what type of language/slangs people use. A backend solution is provided in one embodiment, where the NLP not only collects thousands of comments from the social media forum but then defines much more data such as metadata, synonyms, spelling variations, grammatical variations, sentences, and false positives. For example, “I haven't been thirsty”, means a user is not facing the symptom of increased thirst. If you don't have a mechanism to identify A false positive, then the system will consider that one as the patient is facing the symptom of “Increased thirst”.

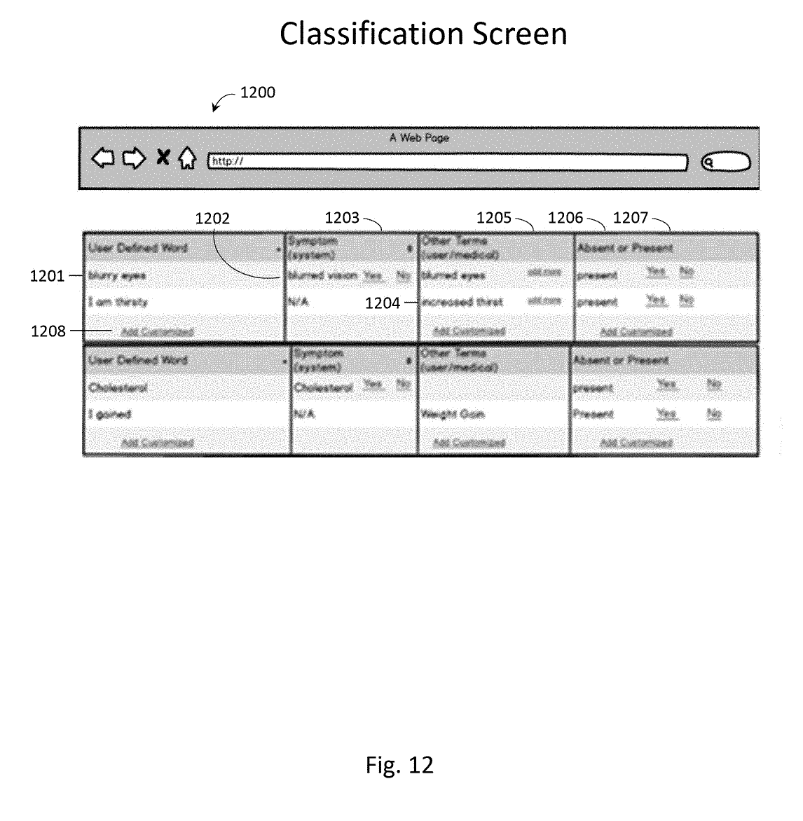

FIG. 12 is a screen shot of a Classification Screen 1200 shows a process for classifying keywords and matching known medical terms, as referenced in FIG. 3, Element 304, according to one embodiment of the present invention. This process incorporates a table with one row per User Defined Word 1201 which represents a sequence of one or more words that are to be classified into normalized data and stored in the DB for further processing. Another column shows a symptom 1202 that is a possible match and shows Yes and No buttons 1203 allowing a user to manually confirm or deny this as a match. In an alternative embodiment, the software automatically makes this determination. Another column shows other terms 1204 that could be used to describe this symptom and shows an Add More button 1205 to allow a user to manually enter more descriptive terms. Another column shows the presence or absence of the symptom 1206 and shows Yes and No buttons 1207 allowing a user to indicate manually if the symptom is present or absent. In one embodiment of the invention, some columns will have a Add Customized button 1208 allowing a user to manually add custom values to the column, which may be processed using NLP, as referenced in FIG. 3, Element 303, transforming input text into key words and matching them with known medical terms. In one embodiment of the invention, a user may be allowed to customize the matching algorithm, by adding or deleting terms and links between them.

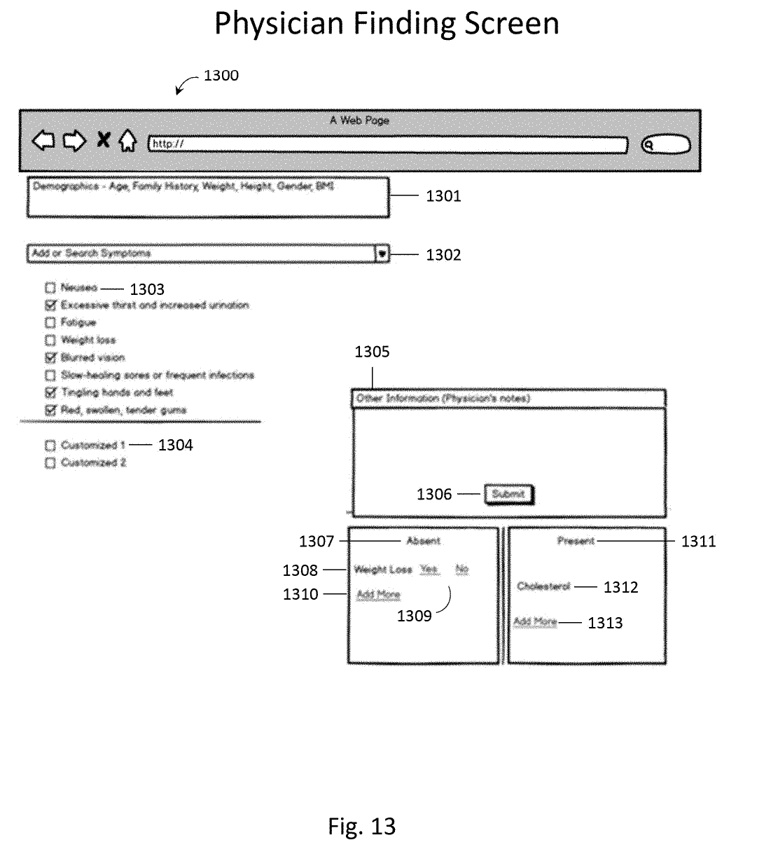

FIG. 13 is a screen shot of a Physician Finding Screen 1300 which may be used by a physician to summarize and edit data for a patient, according to one embodiment of the present invention. Demographics panel 1301 shows the basic demographic information about the patient. Add or Search Symptoms dropdown 1302 allows a physician to add or search existing symptoms from the shared medical knowledge base, referenced as DB in FIG. 3, Element 302. A checkbox list of symptoms 1303 allows a physician to confirm or deny the existence of a specific symptom for the patient. An input method 1304 is included to allow a physician to add customized symptoms for the patient. A physician may enter Other Information 1305 for the patient, such as Physician's Notes.

In one embodiment of the invention, the Other Information field 1305 will be an editable text field containing a Submit button 1306 that allows a user to submit the information when they have finished typing. The Absent panel 1307 shows symptoms 1308 that are absent but suggested as possible matches for the patient and Yes and No buttons 1309 allowing a user to manually confirm or deny each symptom as absent for the patient. The Absent panel 1307 also contains an Add More button 1310 allowing the user to add more symptoms that are absent for the patient. The Present panel 1311 shows symptoms 1312 that are present for the patient. The Present panel 1311 also contains an Add More button 1313 allowing the user to add more symptoms that are present for the patient.

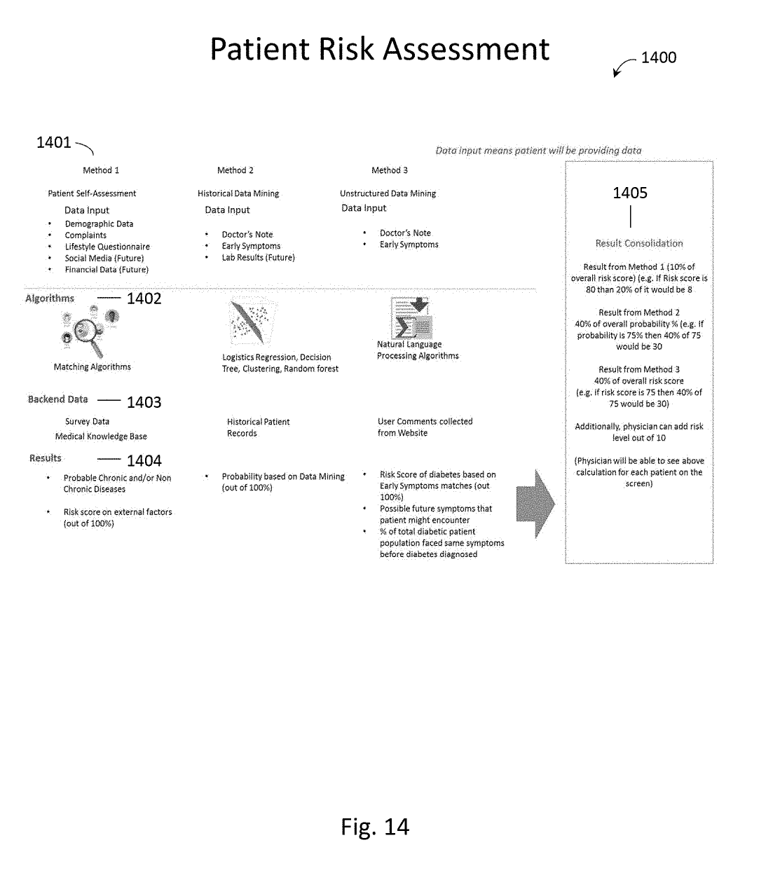

FIG. 14 is a screen shot of a Risk Assessment and Scores screen 1400 which depicts software processes of the present invention which may be used to summarize and explain the calculation of risk scores for a patient, as referenced in FIG. 5, Element 519, according to one embodiment of the present invention.

A table is displayed with one column 1401 for each method used to calculate a risk score included in a risk recommendation. In one row of the table the Algorithm 1402 used to calculate risk scores is displayed. In another row of the table the Backend Data 1403 used to calculate risk scores is displayed. In another row of the table the Results of the risk score calculations are displayed. In the Results Consolidation panel 1405, the consolidated results are displayed for all the listed methods, as referenced in FIG. 2, Element 207. In one embodiment of the invention, a method is based on patient self-assessment, using input data such as demographic data, complaints, questionnaires, social media, and financial data, and using backend data such as survey data and a medical knowledge base, applies a matching algorithm to provide probable and non-probable conditions, and provides a risk score as a numerical percentage. In one embodiment of the invention, a method is based on historical data mining, using input data such as physician's notes, early symptoms, and lab results, and using backend data of historical patient data, applies statistical algorithms such as logistics regression, decision trees, clustering, and random forests to provide a risk score as a numerical percentage. In one embodiment of the invention, a method is based on unstructured data mining, using input data such as physician's notes and early symptoms, and using backend data such as comments collected from a website, applies NLP to provide probable and non-probable conditions, provides a risk score as a numerical percentage, may provide one or more possible future symptoms for a patient to watch for, and a numerical percentage risk based on historical data of actual diagnoses.

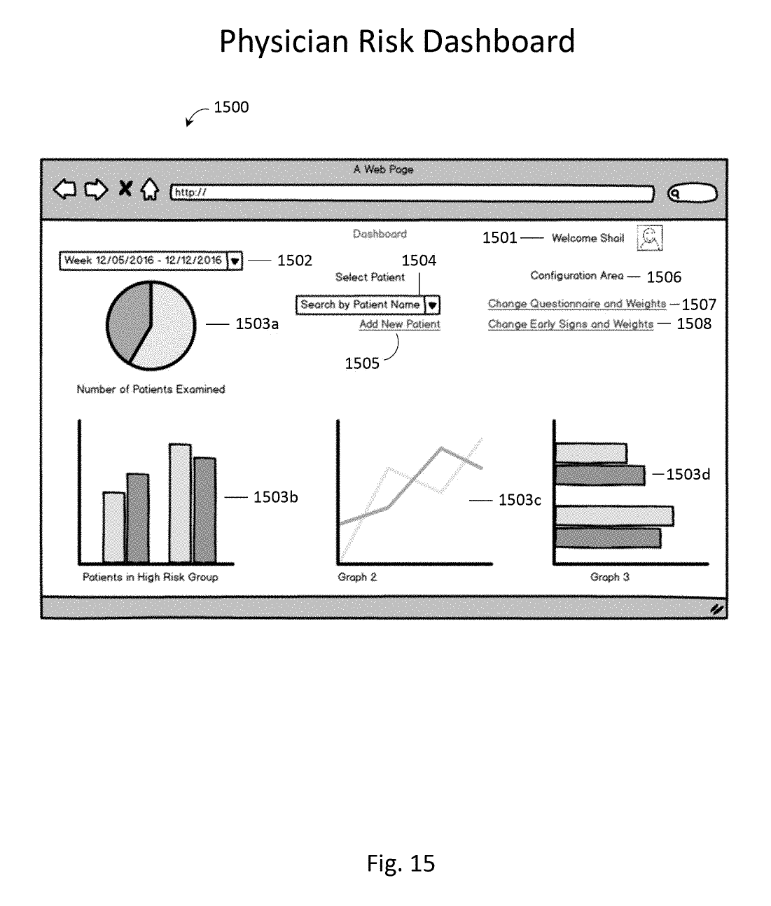

FIG. 15 is a screenshot of a Physician Risk Dashboard screen 1500 which may be used by a physician to summarize and explain the calculation of risk scores for a group of patients, as referenced in FIG. 6, Element 601, according to one embodiment of the present invention. The currently logged in physician is shown 1501. A dropdown 1502 allows the physician to select which data to display from a preset list of time ranges. The selected data is visualized in one or more graphical and formats 1503a, 1503b, 1503c, 1503d with their graphic descriptions listed in FIG. 15. A physician may customize the global way in which risk is calculated using the Configuration Area 1506. A physician changes the questionnaires used and how they are calculated using the Change Questionnaire and Weights button 1507. A physician changes the risk algorithms as referenced in FIG. 14, Element 1402, and how they are calculated using the Change Early Signs and Weights button 1508. A physician searches for a specific patient using a dropdown 1504 or adds a new patient using an Add New Patient button 1505.

In another embodiment, the system provides a physician's sharing functionality under physician dashboard which allows subscribed physicians to share their findings with other physicians who are treating patients in connection with various diseases. This feature helps physicians to provide better treatment to patients based on the outcomes achieved by other doctors. The idea is not to calculate the risk score based on the limited historical patient data from a single physician but to create a pool of data collected and stored from all physicians and then calculate the risk for target patient. Presently, all the solutions available in the market stores data from the subscribed physician or facilities and then provides the risk score for the target patients whereas our solution mines the entire pool of data from all the subscribed facilities and physicians.

For example, say our system has been subscribed by Physician X, Physician Y, and Physician Z.

Number of Patients

Physician X=100

Physician Y=10

Physician Z=1000

Our system will store data for 100 patients for Physician x but then, provide risk score based on the 1110 patients (100+10+1000) of all three Physicians whereas other systems stores data for 100 patients for a subscribed Physician X, Y or Z and then, provide risk score based on the 100 Patients of the Physician X, Y or Z only. The present system monitors data for more patients and then identifies accurate pattern of risk of diseases.

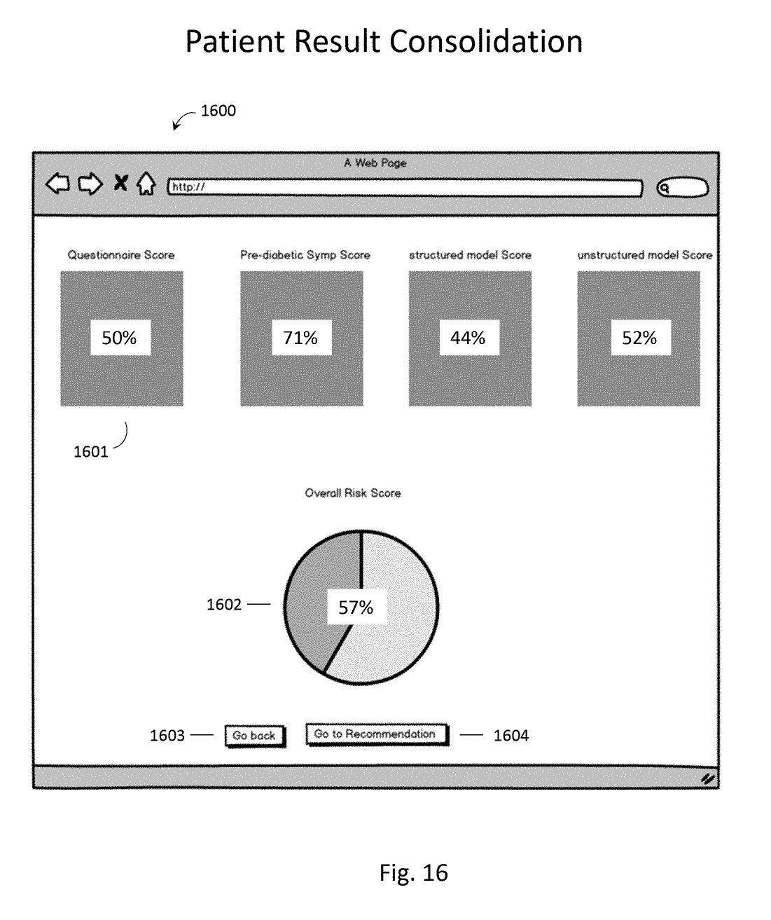

FIG. 16 is a screen shot of a Patient Result Consolidation screen 1600 which summarizes and visualize risk scores for a patient, as referenced in FIG. 14, Element 1405, according to one embodiment of the present invention. This embodiment demonstrates a risk recommendation, as previously discussed. A score is shown for medium implemented to gather symptoms and data and each method used to calculate patient risk, as referenced in FIG. 14 Element 1401. In one embodiment of the present invention the Overall Risk Score 1602 is shown in a graphical format. In another embodiment of the present invention the Overall Risk Score 1602 is shown in a numerical text format, or both as depicted here. A user goes back to the previous screen using a Back button 1603. A user goes to another screen showing the Patient Recommendation as referenced in FIG. 5b, Element 520.

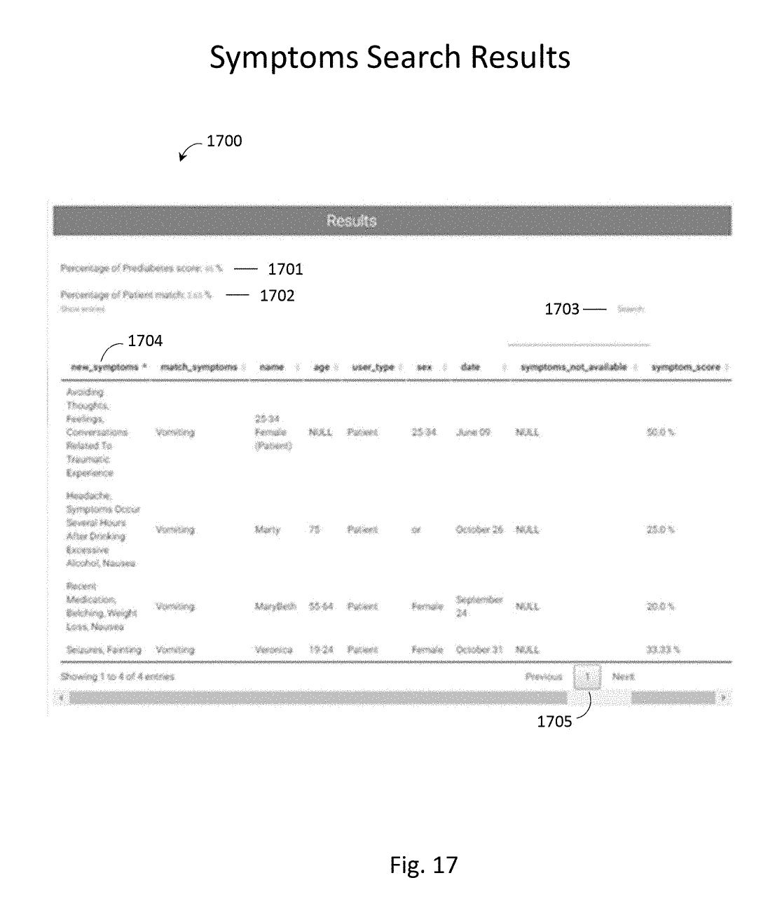

FIG. 17 is a screen shot of a Symptoms Search Results screen 1700 which displays search results for a symptom, as referenced in FIG. 13, Element 1302, according to one embodiment of the present invention. The associated weighting percentage for this symptom when calculating risk scores is displayed 1701. The percentage of patients matching this symptom is also displayed 1702. A user may create a new search for a symptom using a search box 1703. After each search, a table 1704 is displayed with one row for each returned result from the requested search. Navigation controls 1705 allow a user to browse the table 1704.

In this embodiment, the system analyzes information and data submitted from other patents. Also anonymous data may be shared by physicians and between physicians to add to the system analytics and to increase the data in and to update the data in the knowledgebase.

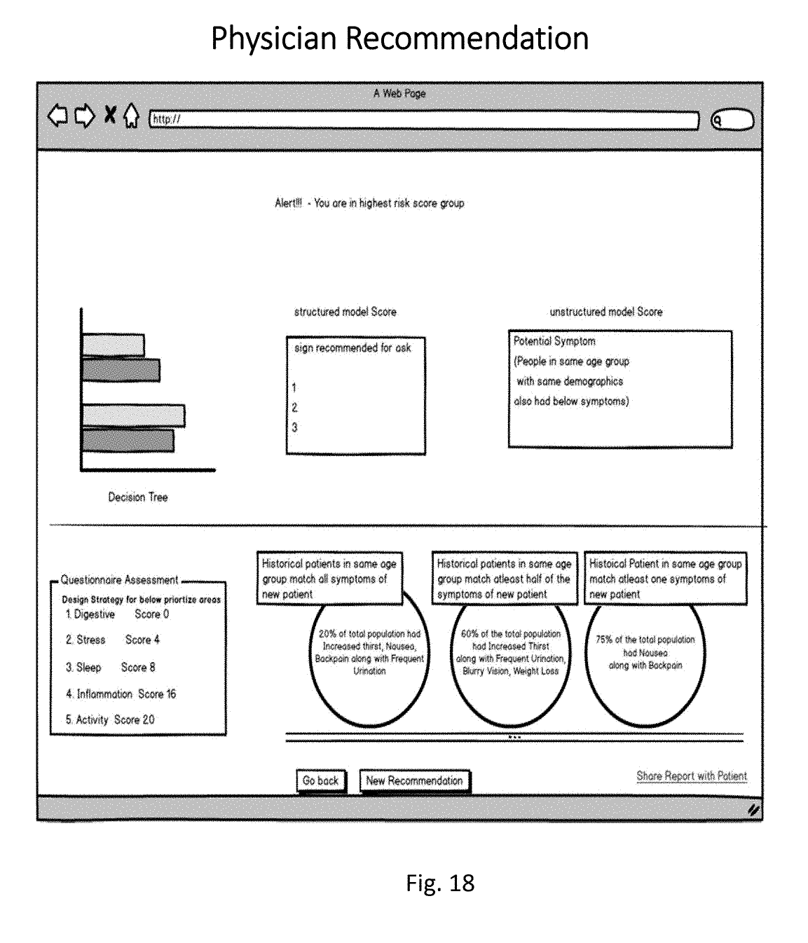

FIG. 18 shows a Physician Recommendation screen designed to assist the physician in developing a recommendation for the patient. The physician may view calculated risk scores. Develop a recommendation that includes future risk symptoms, historical available data regarding similar patients, accessed via the system or from publically available data, and provides a recommendation or treatment which may be accessible to the patient via the patient dashboard or provided directly. Generally, most or all functions and data retrieved from and provided to a patient is accessed via various interfaces accessed through the patient dashboard. Similarly, the physician functions are accessed via the physician dashboard, although the physician will have access to the patient dashboard.

We claim:

1. A system for identifying and predicting risk level of medical conditions of a patient comprising:
a web server for hosting and serving web pages to network connected nodes or devices upon request;
a database server connected to the web server for storing data related to one or more patients;
an application to access and process data from the database server; and
a visualization tool stored on the database server, the visualization tool configured to display graphics and data related to medical conditions of the one or more patients.

2. The system of claim 1, wherein the patient data is received by the database server through:
(i) information provided by the patient on the web server;
(ii) information provided by the physician on the web server;
(iii) information gathered from the available research and literature data; and
(iv) information scraped from public sources about the patient.

3. The system of claim 1, wherein the database server includes a Natural Language Processing module to transform scraped and input data into medically usable data.

4. The system of claim 3, wherein the Natural Language Processing module includes a computerized visual processing module for generating medical data using facial recognition, pattern recognition and item identification.

5. The system of claim 1, wherein the visualization tool provides a risk score and recommendation to the patient and the physician.

6. The system of claim 2, wherein the database server further includes data from comparison groups for other patients and physicians stored in the database server.

7. A method for identifying and predicting risk level of medical conditions of a patient comprising:
gathering information associated with the patient from one or more data sources;
storing the information in a database server;
processing the information from the database server; and
displaying data and graphics related to medical conditions of the one or more patients on the visualization tool.

8. The method of claim 7, wherein the processing of information is done by a Natural Language Processing module.

9. The method of claim 8, wherein the Natural Language Processing module can scrape and process information from publicly available data for patients and physicians.

10. The method of claim 7, wherein the visualization tool displays one or more of a preliminary assessment report, risk assessment report, patient notes report, physician recommendation report and patient result consolidation report.
